(19) United States
(12) Patent Application Publication
(10) Pub. No.: US 2013/0102948 A1
(43) Pub. Date: Apr. 25, 2013

(54) PORTABLE BLOOD FILTRATION DEVICES, SYSTEMS, AND METHODS

(71) Applicants: Ilan K. Reich, New York, NY (US); Edward F. Leonard, Bronxville, NY (US); Fred Mermelstein, Newton, MA (US)

(72) Inventors: Ilan K. Reich, New York, NY (US); Edward F. Leonard, Bronxville, NY (US); Fred Mermelstein, Newton, MA (US)

(21) Appl. No.: 13/641,559

(22) PCT Filed: Mar. 24, 2011
(86) PCT No.: PCT/US11/29854

§ 371 Date: Dec. 26, 2012

(30) Foreign Application Priority Data
Apr. 19, 2010 (US) 61325765

(51) Int. Cl. A61M 1/36

(52) U.S. Cl. CPC A61M 1/3621
USPC 604/6.09; 210/649; 210/641; 210/638; 210/634

(57) ABSTRACT

A blood filtration device, system, and method that can and selectively remove or reduce an unwanted, in certain cases unknown, substance from a patient's blood stream. More specifically, a specific size or size range of unwanted substance is selectively removed. The unwanted substance includes one or more of a pathogen, a toxin, an activated cell, and an administered drug. The device and system employ a microfluidic separation device that minimizes thrombogenesis and can permit the use of anticoagulants to be avoided. The device or system can be portable and can include its own power supply. Sensors in the system may monitor for the presence and/or concentration of unwanted species including pathogens or drugs and invoke a blood cleansing process responsively to the sensor signals in closed loop control process. The control may combine the infusion of therapeutic agents into the blood of a patient as well.

### CROSS-REFERENCE TO RELATED APPLICATION

The present application claims the benefit of U.S. Provisional Application No. 61/325,765 filed Apr. 19, 2010, the entire content of which is hereby incorporated by reference.

### FIELD

In general, the disclosed subject matter involves selective removal of unwanted components in a patient's blood including, but not limited to, pathogenic particles, chemicals, and other particles.

### BACKGROUND

A localized infection, such as at a wound site, if not promptly and properly diagnosed and treated can oftentimes progress to a serious and overwhelming blood infection caused by the presence of pathogenic microorganisms or their toxins in the blood stream. Such blood infection is commonly known as sepsis and may lead to limb amputation, organ dysfunction or failure, or even death.

Sepsis diagnosis has typically involved using a relatively small percentage of a patient's blood (e.g., 0.1% of less) for a culture or molecular analysis. Further, even if detected, treatment has typically involved a “broad-brush” approach, whereby a non-specific antibiotic is used. However, in some cases, the non-specific antibiotic may not be effective treatment against an infecting pathogen. Moreover, frequent use of non-specific antibiotics can also promote antibiotic-resistant bacteria.

In another context, the low therapeutic index and high toxicity of many chemotherapeutic agents is exacerbated by their long term persistence in the human body. In particular, myelosuppression can be an undesirable side effect of virtually all chemotherapy treatments. Many protein-bound chemotherapy agents have extended half-lives relying on elimination by the liver or kidney, and there is often cumulative toxicity based on the cumulative effects of the treatment regimen.

SUMMARY

The Summary describes and identifies features of some embodiments. It is presented as a convenient summary of some embodiments, but not all. Further the Summary does not necessarily identify critical or essential features of the embodiments, inventions, or claims.

Generally speaking, included among embodiments described herein is a blood filtration device or system that can selectively remove or reduce unwanted substances or components from a patient's blood stream, and thereafter replenish the patient with “cleaned” blood. In various embodiments, the unwanted substance may be unknown prior to operation of the blood filtration device or system. Such systems and devices can be used to treat infections such as local or blood infections described above or as part of chemotherapy treatment. The system can also be used to allow a broader range of pharmacokinetic options such as varying the concentration of antibiotics or other agents using cycles of infusion and removal of such infusates. The system can be used to dialytically remove target substances from blood, such as bacteria, while minimizing the exposure of blood to thrombogenic surfaces thereby, in embodiments, eliminating the need for anticoagulants.

Embodiments described herein can be configured as mobile systems or devices, such that they can be moved, for example, from room to room in a hospital. Embodiments can be deployed in mobile medical units, such an ambulance or medivac helicopter, for use by emergency or military personnel. Alternatively, the embodiments can be configured as a portable device that emergency responders or military personnel can carry to a treatment site. In yet another alternative, embodiments can be configured as substantially fixed units with appropriate fluid conveyances and/or storage vessels for transporting to a target or treatment location. In still another alternative, embodiments can be fixed at a particular location, such as a floor or wing of a medical treatment facility. In some or all embodiments the device or system can contain its own power supply (e.g., a battery or batteries), which can serve as the system or device primary or back-up power supply.

According to embodiments, the disclosed subject matter includes any devices and or systems configured to implement any of the methods described herein.

### BRIEF DESCRIPTION OF THE DRAWINGS

Embodiments will hereinafter be described in detail below with reference to the accompanying drawings, wherein like reference numerals represent like elements. The accompanying drawings have not necessarily been drawn to scale. Any values dimensions illustrated in the accompanying graphs and figures are for illustration purposes only and may not represent actual or preferred values or dimensions. Where applicable, some features may not be illustrated to assist in the description of underlying features.

FIG. 1 shows an arrangement for plasma extraction by microfluidic separation with small particle/molecule removal by plasma filtration or selective removal of any species by adsorption.

FIG. 2 shows an arrangement for plasma extraction by microfluidic separation with large particle/molecule removal by plasma filtration.

FIG. 3 shows an arrangement for plasma extraction by microfluidic separation with middle size particle/molecule removal by cascade filtration.

FIGS. 4 and 5 show arrangements for plasma extraction by microfluidic separation with plasma replacement.

FIG. 6 illustrates a dialysis based plasma purification system in which flow selector valves, controls, and other elements that are usable in any of the embodiments are employed to permit the closed loop cycling of cytoplasmic body-free blood fractions in a microfluidic separation module during infusion and treatment intervals in which blood is not being cleansed.

FIG. 7 shows a control system with associated controllers and sensors.

FIG. 8 shows an arrangement for plasma extraction by microfluidic separation with middle size particle/molecule removal by cascade filtration without implementation of drug infusion.

FIGS. 9A and 9B schematically illustrate the application of alternative cleansed plasma return line configurations with respect to the operation of the microfluidic separation module.

FIG. 10 illustrates a field application of a portable device for treatment of sepsis, for example, treatment of wounded personnel in a battlefield environment.

FIG. 11 illustrates embodiments in which one or more secondary treatment components are selectively implemented.

### DETAILED DESCRIPTION

The detailed description set forth below in connection with the appended drawings is intended as a description of various embodiments of the disclosed subject matter and is not intended to represent the only embodiments in which the disclosed subject matter may be practiced. The detailed description includes specific details for the purpose of providing a thorough understanding of the disclosed subject matter. However, it will be apparent to those skilled in the art that the disclosed subject matter may be practiced without these specific details. In some instances, well-known structures and components are shown in block diagram form in order to avoid obscuring the concepts of the disclosed subject matter.

Patent application Ser. No. 11/814,117 (Pub. No. 2009/0139931) filed May 22, 2007 (hereinafter “the '117 application”), which was attached in Appendix A in the above-referenced provisional patent application, and which is hereby incorporated by reference in its entirety into the present application, discusses the use of a secondary treatment device to remove waste from plasma such as for treatment of end stage renal disease. In the disclosure of the '117 application, a microfluidic separation device is employed to extract a cytoplasmic-body-free fraction of whole blood extracted from a patient, hereafter referred to as plasma. The filtering of toxins from the plasma may be done in an extracorporeal treatment process with whole blood entering the microfluidic separation device, and splitting into two streams, one of a plasma fraction (plasma and components to be removed), and the other whole blood with the plasma fraction removed. The latter may be directly returned to the body while the plasma fraction is passed through a filter, exposed to an adsorbent or other substance removal device such as a deionization filter (e.g., cation-ion exchange) so as to remove undesired components or portions thereof. For example, water volume may be reduced by ultrafiltration. A combination of removal mechanisms may be used as well. The treated plasma is then returned to the patient. Also incorporated by reference in its entirety into the present application is U.S. Pat. No. 7,588,550 which describes related technology.

Other extraction mechanisms known now or which may later be discovered may be used as a secondary processor in any of the embodiments described herein to create alternative embodiments. For example, newly discovered engineered tissues (so called organs on a chip) may be used or centrifugation or processes that employ binding agents such as opsonins.

In the disclosed subject matter, embodiments of microfluidic separation components of the treatment devices and systems described above extract plasma containing targeted substances from whole blood. The microfluidic separation device may be combined with a secondary treatment device that removes the target substances from the plasma. The target substances may include particles such as pathogens such as bacteria, middle or large molecular-weight proteins, metabolic solutes or drugs, for example. In embodiments, substances of smaller size or lower molecular weight than the target substance may be returned to the patient. In such embodiments, substitution fluid may be administered, for example to make up lost volume and/or lost precious blood substances such as albumin. The secondary separation device may employ any suitable mechanism for removing target substances from the blood, including adsorption, double filtration with a membrane, single stage filtration with a membrane, centrifugation, etc. Cleansed plasma may be returned to the body.

The use of microfluidic separation devices for clearing substances, such as pathogens, toxins, or drugs from a patient's blood may offer at least these benefits over existing technologies for removing pathogens, their toxins, or other toxins (including administered drugs, such as antibiotics or chemotherapy drugs and/or medicaments or other therapeutic agents) from blood.

First, host cytoplasmic body depleted fractions may be extracted and replaced relatively rapidly and with less exposure to artificial materials because of the properties and capabilities (e.g., flow rate, biofouling resistance, etc.) of the microfluidic separation module as a plasma separation device. Note the term “host” is used here to indicate that the cytoplasmic bodies referenced are non-contaminating elements such as sepsis-causing bacteria that are to be removed. In the remainder of the instant specification, references to the cytoplasmic body free fraction indicate it is free of host substances but not necessarily free of contaminating particles.

Second, the volume of plasma that needs to be removed can be reduced due to the small extracorporeal volume of the microfluidic separation module. Third, there is a lower latency between the time plasma is circulating in the patient and the time plasma is extracted, allowing for real-time detection of pathogens, toxins, or drugs in the plasma fraction. Signal levels for such real time detection can be higher for these substances circulating in the plasma fraction of the blood because of the higher concentration as compared to whole blood and the reduced diffusion caused by the presence of cytoplasmic bodies. Fourth, the reduced exposure to artificial surfaces provided by the microfluidic technology may compensate the use of less biocompatible materials or longer exposure time to materials with low biocompatibility. Though not relevant or minimized, should inadvertent removal of essential blood components occur, any such effects to the patient can be negated or reduced because of the aforementioned relatively lower time period in which blood components are external to the patient.

Additionally, the use of a microfluidic separation module to separate plasma from whole blood prior to applying a selective filtration method to the plasma, besides having the other benefits described in the patent-related documents herein incorporated by reference, also concentrates the target particles or molecules relative to whole blood, since the latter are confined to the plasma portion of whole blood. Also, for the same reason, the diffusion path length for solutes and particles, and thus the diffusivity thereof, may be enhanced relative to that in the presence of cytoplasmic bodies. This may increase the effectiveness of sensors that detect analytes. It may also increase the efficiency of secondary separation, reduce the fouling of filters used in such secondary separation, reduce treatment time, and reduce the amount of whole blood required to be processed for a given amount of blood cleansing. For all embodiments disclosed, the detectors/sensors which detect undesired species in blood may be embodied in small microfluidic separation devices which extract plasma in very small amounts for sampling purposes only.

For instance, the concentration of drug in the plasma according to embodiments may be about twice as high as that in whole blood. The higher concentration and the lack of cells may improve the signal for concentration monitoring systems as well as improve the removal rate and/or efficiency of adsorbent and other secondary treatment devices. Removal of drug from cell free plasma may also permit the use of less biocompatible materials, such as certain adsorbents.

Other benefits of the microfluidic technology described herein and in the patent-related documents discussed and incorporated by reference herein will also be appreciated.

According to embodiments, microfluidic separation of plasma is combined with further processing of the plasma to remove particles (or molecules) in a predefined size range by passing the plasma through a double filtration system. That is, target molecules are discriminated from the plasma by a slicing cascade of membrane filters. The purified plasma can then be returned to the patient or the microfluidic separation device (which may ultimately return the purified plasma to the patient).

According to embodiments, microfluidic separation of plasma is combined with further processing of the plasma to remove particles (or molecules) of a predefined species by exposing the plasma to an adsorbent or other removal mechanism. Target molecules are discriminated from the plasma by chemical or physical interaction with the adsorbent. The purified plasma can be returned directly to the patient or the microfluidic separation device (which may ultimately return the purified plasma to the patient).

According to embodiments, microfluidic separation of plasma is combined with further processing of the plasma to remove particles (or molecules) of large size by passing the plasma through a filter membrane. A filtrand stream containing large molecules blocked by the filter is discarded while the filtered stream is returned to the microfluidic separation device or to the patient.

According to embodiments, the disclosed subject matter includes a method for extracorporeal treatment of blood to remove a target substance. The method includes removing blood from a patient and passing the blood through a microfluidic channel, the passing including diffusing the target substance into a cytoplasmic body-free blood fraction using a microfluidic separation module that employs microfluidic channels and microsieve wall filters. The method further includes removing the cytoplasmic body-free fraction from the microfluidic channel and extracting at least some of the target substance from the removed cytoplasmic body-free fraction and returning the resulting cytoplasmic body-free fraction to the microfluidic channel.

The extracting may include cascade filtration using multiple membranes having different pore sizes to select and filter out a target particle size range. The extracting may include adhering the target to an adsorbent. The method may include monitoring an amount of the target substance in the body of a patient and controlling an administration of the target substance, or a precursor thereof, responsively to the monitoring. The method may include binding the target substance to another substance to form an aggregate particle, the extracting including extracting the aggregate particle. The aggregate particle may have a higher binding affinity for an adsorbent and the extracting includes exposing the aggregate particle to the adsorbent. The method may include monitoring an amount of the target substance in the body of a patient and controlling an administration of the target substance, or a precursor thereof, according to a time concentration integral limit is not exceeded. The method may include monitoring an amount of the target substance in the body of a patient and controlling an administration of the target substance, or a precursor or metabolite thereof, responsively to predictive model of an elimination rate of the target substance from the patient by endogenous pathway. The patient may have a faulty endogenous elimination capacity and the method may include identifying the patient as a candidate for the method based on the faulty capacity. The target substance may be a result of a drug overdose.

According to embodiments, the disclosed subject matter includes a method for extracorporeal treatment of blood to remove a target substance. The method includes removing blood from a patient and passing the blood through a microfluidic channel which separates plasma from a host cytoplasmic body-rich fraction of the whole blood. The method further includes removing and discarding the cytoplasmic body-free fraction from the microfluidic channel. The method further includes returning fresh plasma or components thereof, such as albumin, to the microfluidic channel.

In any of the embodiments, the target molecules may be bound chemically to a molecule of a predefined size to form particles or molecules having a predefined aggregate size or having predefined chemical properties, such as a binding affinity, which aggregate may be discriminated by any of the filtering processes discussed above. The filter cascade may be designed to remove an aggregate particle (i.e., the target bound to the predefined particle or molecule) rather than the target molecule alone. According to embodiments, the disclosed subject matter may be applied to perform hemoperfusion through carbon, or reticular filter columns may be used; plasmapheresis or apheresis with plasma replacement; plasmapheresis with plasma perfusion through sorbents which bind to proteins, bilirubin and/or aromatic amino acids; standard hemodialysis, standard hemodialysis with an amino acid dialysate and plasma exchange; high permeability hemodialysis or dialysis with charcoal-impregnated or anion-exchange membranes.

Examples of target substances for removal include bacteria, viruses, toxins and biomolecules, and patient cells. For instance, small molecular toxins and protein-bound molecules or heavy molecules associated with liver disease may be removed. Other examples include protein-bound ammonia, phenols, mercaptans; fatty acids, aromatic amino-acids, salts. Also, larger molecules such as and bacterial particles and endotoxins. Examples of target bacteria include Y pestis, F. tularensis, B. anthracis, Streptococcus pneumoniae, K pneumonia, A. calccoaceticus-baumannii complex, S. aureus, P. aeruginosa, etc. Examples of target viruses include Hepatitis C, Influenza, smallpox, HIV, viral hemorrhagic fevers, etc. Target toxins and biomolecules include Aflatoxin, amatoxin, alpha toxin, botulinum toxin, endotoxin, ricin, Shiga toxin, tetanus toxin, cytokines, etc. Target patient cells include activated platelets, activated neutrophils, lymphocytes producing pro-inflammatory cytokines, etc.

Chemotherapy agents may also be targets for removal. For example, a secondary separation method may be to use a membrane dialyzer with adsorbent particles in the dialysate, which removes toxins exchanged across the membrane as described in U.S. Pat. No. 5,277,820. In such an embodiment, a stream of plasma is generated using a microfluidic separation device and subjected to a secondary treatment process, as described in U.S. Pat. No. 5,277,820. Any of the other processes for extracting target substances may be used to remove chemotherapeutic agents from the plasma. The entire content of U.S. Pat. No. 5,277,820 is hereby incorporated by reference into the present application.

Also included among embodiments described herein is a systemic oncology drug delivery system which increases the range and precision of control of drug delivery and drug elimination from the body of the patient. This may be used to provide, for example, a higher maximum tolerated dose (MTD) for certain agents. In embodiments, systems can include an infusion pump for drug administration, an extracorporeal blood purification circuit that concurrently and efficiently removes protein-bound toxins, and an adaptive control system to monitor and regulate delivery and removal.

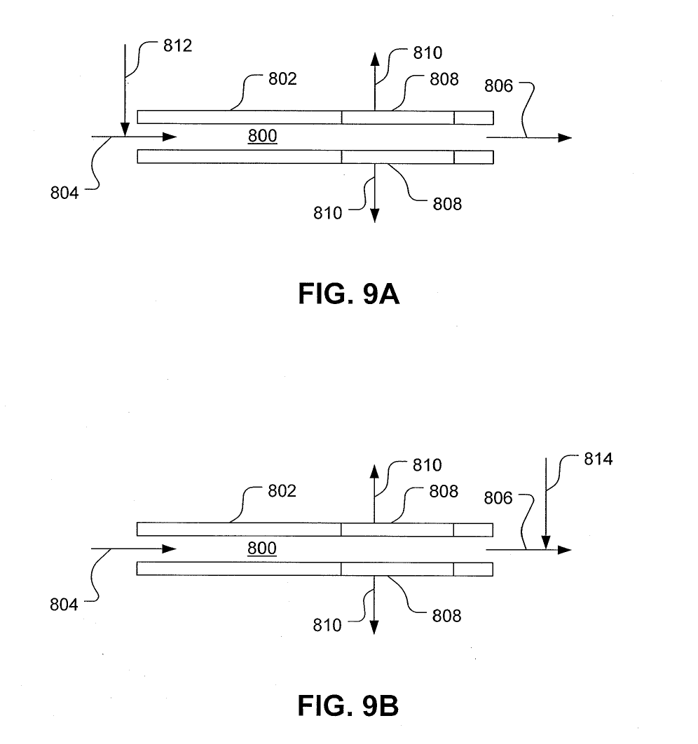

In embodiments, blood is removed from a patient's body via a device or system implementing a closed-loop fluid path. Plasma is first separated from blood using a microfluidic separation module. The module can operate to generate an albumin-rich cell-free stream, which is then passed through a sorbent which separates the unwanted species from albumin. The purified plasma returns to the patient's blood through the microfluidic separation module or directly by infusion (as discussed for example with reference to FIG. 10).

Using embodiments described herein, a patient at risk for sepsis can be connected to a portable device monitored by slow removal and testing of plasma fraction and rapidly treated by removal of bacterial particles. The embodiments can be combined in a system that also provides for the automated or operator controlled administration of therapeutic agents such as antibiotics.

Using the embodiments described herein, the efficacy of chemotherapy drugs may be modified by employing systems for apheresis or chemofiltration of plasma extracted by a microfluidic separation module. This may be employed as a treatment modality for chemotherapeutic agents, which are introduced at high levels and then removed. Area under the curve toxicity can be reduced while spiking the blood levels of one or more therapeutic agents over respective time intervals, thereby providing broader range of pharmacokinetic options to a treating physician. By reducing the drugs or their metabolites that are simply circulating in the blood and which have not bound to tumor cells, their toxic effects may be controlled. The patient's own natural elimination mechanisms can thus be augmented. In embodiments described herein, real time automatic adaptive control is described in combination with chemofiltration which may provide still more pharmacokinetic options for treatment.

A specific therapy employing a therapeutic agent, such as used in chemotherapy, may be provided according to the disclosed subject matter. Some therapeutic agents have toxic effects. In many cases, the dosages required for treatment are toxic. In the therapy embodiment, a therapeutic agent is introduced into the blood supply at a first time and removed later or simultaneously at a different point from that of infusion to augment the patient's own natural elimination mechanisms, if one exists, or to provide an elimination mechanism, if none exists or is impaired (for example, due to renal or hepatic insufficiency). This may reduce the burden on organs such as kidneys and liver and reduce the toxicity on healthy cells which are susceptible to being destroyed during and after chemotherapy treatment (e.g., bone marrow). Also agents such as imaging contrast agents or other diagnostic materials that are useful for a period of time but which burden the elimination capabilities of weakened patients can be spared from iatrogenic problems. Also blood levels of other drugs such as antibiotics may be “profiled” in a similar manner.

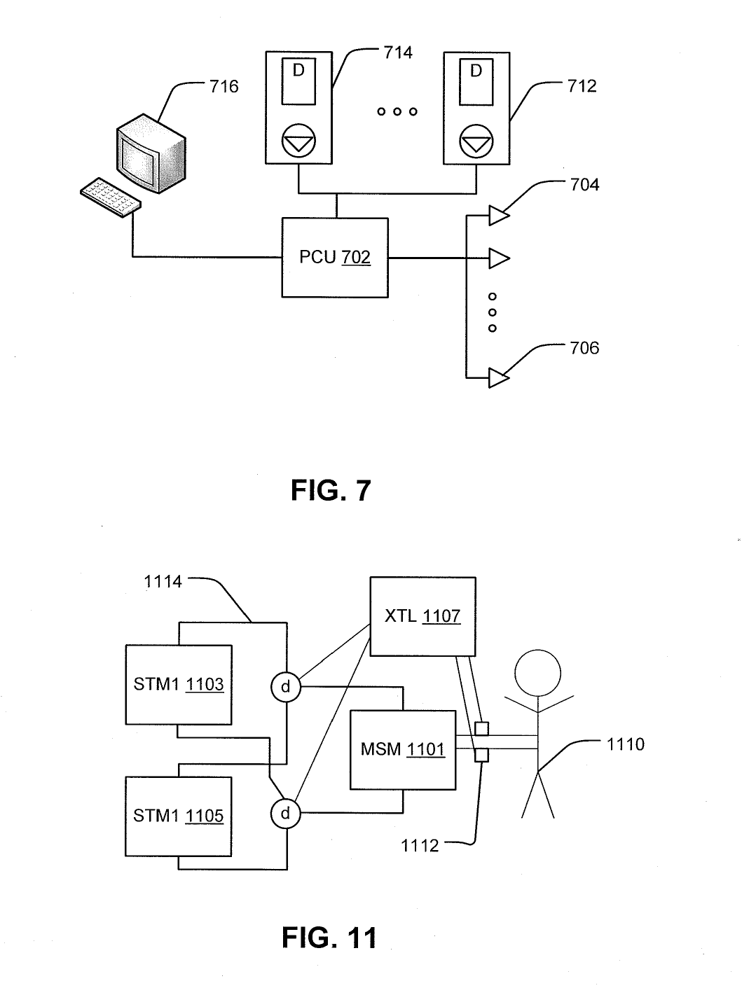

As described with reference to FIG. 8 of the '117 application, despite flow blood and extraction fluid in microfluidic channels in a co-flowing manner, it is possible to arrange multiple channels in series to approximate a counterflow configuration with a higher extraction efficiency. Assume complete equilibration of the mass of the drug between the extraction fluid and the blood in the microfluidic channels. The microfluidic channels (or sub-units) are series connected by the inlet and outlet blood ports of the channels. The plasma ports (the plasma being the extraction fluid that flows through the secondary treatment component to remove the drug) are connected in a reverse sequence so that plasma enters the last microfluidic channel sub-unit through which the blood passes. Assume there are N sub-units, and the concentration of drug in the blood is X while the concentration of drug in cleansed plasma is Y. Then the concentration of the drug leaving the final microfluidic channel will be

$$1 - \frac{( X - Y )}{( N + 1 )} .$$

The approximation is based on the assumption that the volume of blood and plasma in the microfluidic channel is approximately the same in each microfluidic channel.

In embodiments, a feedback loop controls the administration of a drug, for example, a chemotherapeutic agent or antibiotic. Embodiments of the disclosed methods can be applied to remove the drug or other unwanted component. The control may be configured to provide real time feedback control based on total load of the target substance in the body of a patient. The control variable may be obtained from a real time assay, for example as described in International Application No. PCT/U.S. 2010/031600 corresponding to International Publication No. WO 2010/123819, the entire content of which is hereby incorporated by reference. Alternatively, the control variable for feedback control may be a predictive model of the total amount of drug in the patient. The control system logic may be used to manage the infusion rate of the drug so as to maintain a desired profile of drug dose and/or toxin level. Control based purely on pharmacokinetic models may not be as personalized for a given patient and may not account for unique patient responses. Real-time feedback offers a much more precise way of managing dose and toxicity.

Embodiments may include detection and regulation of undesired blood components such as pathogen levels. These embodiments may also include detection and regulation of levels of therapeutic agents that change the levels of the unwanted substances and the regulation of the blood levels of the therapeutic agents themselves. Thus in all of the embodiments described herein, multiple types of detectors may be employed such as one indicating the presence of infection and another for indicating the blood levels of a treatment agent. The system may be configured to regulate the levels of both. The latter capability may be provided by the use of combinations of treatment components (secondary treatment devices that remove target substances from extracted plasma) which are selectively switched in and out of the plasma loop responsively to the detected levels of species in the blood or plasma.

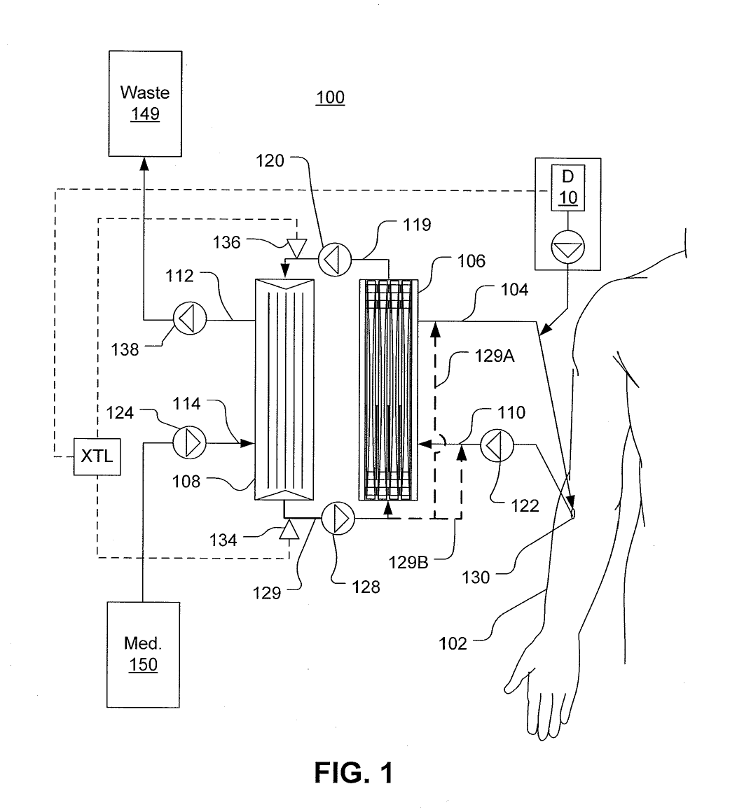

FIG. 1 shows a chemofiltration system 100 that performs plasma extraction by microfluidic separation. The system achieves small particle/molecule removal by plasma filtration or selective removal of any species by adsorption. Blood is drawn from a patient 102 via an arterial line 104 and passed through an arterial line and into a microfluidic separation module 106 as described in the '117 application, which is hereby incorporated by reference as if set forth in its entirety herein. Blood is returned to the patient via a venous line 104. The microfluidic separation module 106 removes a cytoplasmic-body-free fraction of the blood, exchanges components by diffusion, or a combination of the two. The cytoplasmic-body-free fraction of the blood passes through a supply line 119, impelled by a pump 120, into a filter 108 where a target component is removed by some process such as dialysis, plasma filtration, or adsorption. The cleansed cytoplasmic-body-free fraction of the blood is returned to the microfluidic separation module 106 via a return line 129, 129A, or 129B driven by a pump 128.

Lines 129, 129A, and 129B represent return line configuration for respective alternative embodiments. The cleansed blood fraction can be returned to the patient at various points in the system including, directly to the microfluidic channel via line 129 as described, for example, in the '117 application. Alternatively, the cleansed blood fraction is returned by line 129A to the patient venous line 104. Another alternative embodiment returns the cleansed fraction via line 129B to the arterial line 110.

In an embodiment, the filter 108 is a dialyzer, in which case a supply of dialysate 150 is pumped through the dialyzer (filter 108) via a supply line 114 and pump 124, where it passes along a filter to exchange components with the cytoplasmic-body-free fraction of the blood, is recovered, and then discarded (149) via discharge line 112 and pump 138. Detectors 134 and 136 detect an amount of a target substance in the supply and discharge lines 114 and 112, allowing a controller XTL to determine an amount of a target substance being removed.

In any of the embodiments of FIGS. 1-6 and 8, the detectors 134 and 136 can be placed on plasma lines, blood lines, or dialysate lines as desired. Alternatively, detectors 134 and 136 can be integrated in fluid-conveying components such as the inlet and header chambers of the filter 108 or similar components. For example, they may be located on plasma lines 119 and 129 or blood lines 104 and 110, because the plasma and fluid may have higher toxin concentrations and therefore provide a stronger signal. However, using the dialysate lines 119 and 129 can have an advantage if the detectors 134 and 136 provide a more reliable signal based on a lower viscosity fluid such as dialysate. This may be done in near real time by a lab on a chip assay device, for example, or by some other type of sensor. The controller may use the rate of removal of the target to control the infusion pump 10 which infuses a drug or medicament into the venous line 104 and thereby into the patient 102.

Examples of substances that may be infused and then recovered also include imaging contrast agents, diagnostic agents, and treatment drugs such as chemotherapeutic agents for cancer treatment.

In an embodiment, the infusion pump is used to infuse a patient with a therapeutic agent that has some known toxicity. The chemofiltration circuit may be primed and the patient access established and patency of the access maintained until a time governed by the controller XTL according to a stored treatment plan. The controller XTL may then stop the infusion and after a second interval, start the chemofiltration system at a rate responsive to the treatment plan. The treatment plan may provide for a specific time-varying concentration of drug in the patient by controlling both the infusion system 10 and chemofiltration system 100 thereby providing flexibility to a treating entity.

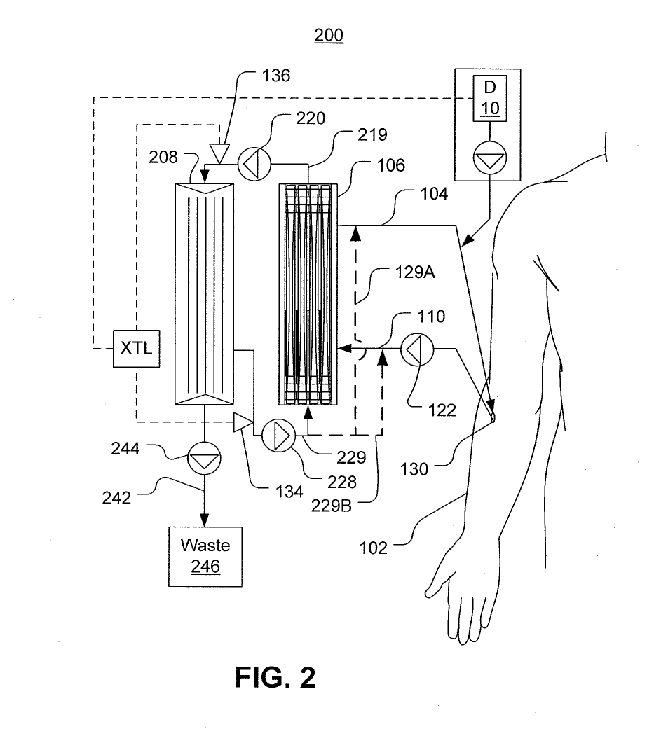

FIG. 2 shows a system 200 for plasma extraction by microfluidic separation with large particle/molecule removal by plasma filtration. Blood is drawn from the patient 102 and passed through an arterial line 110 and into a microfluidic separation module 106 as described in the '117 application. Blood is returned to the patient 102 via the venous line 104. The microfluidic separation module 106 removes a cytoplasmic-body-free fraction of the blood, exchanges components by diffusion, or a combination of the two. The cytoplasmic-body-free fraction of the blood passes through a supply line 219, impelled by a pump 220, into a filter 208 where a target component is removed by filtration with a pore size selected to block the target molecule and pass other plasma components. The cleansed cytoplasmic-body-free fraction passing through the membrane of the filter 208 is returned to the microfluidic separation module 106 via a return line 229 driven by a pump 228. A pump 244 may be used to draw the target particle in a filtrand stream which may be discarded 246. As described above, alternative embodiments of return lines 229A and 229B may be provided instead of return line 229.

In an embodiment, the filter 208 has a membrane whose pores are large enough to pass albumin molecules and small enough to block larger particles that are the target particle or are bound thereto.

Detectors 134 and 136 detect an amount of a target substance in the supply and discharge lines 219 and 229, allowing a controller XTL to determine an amount of a target substance being removed. This may be done in near real time by a lab on a chip assay device, for example, or by some other type of sensor. The controller may use the rate of removal of the target to control the infusion pump 10 which infuses a drug or medicament into the venous line 104 and thereby into the patient 102. Examples of substances that may be infused and then recovered also include imaging contrast agents, diagnostic agents, and treatment drugs such as chemotherapeutic agents for cancer treatment.

In any of the embodiment, the infusion pump may be used to infuse a patient with a therapeutic agent as described above, under control of the controller and according to a treatment plan.

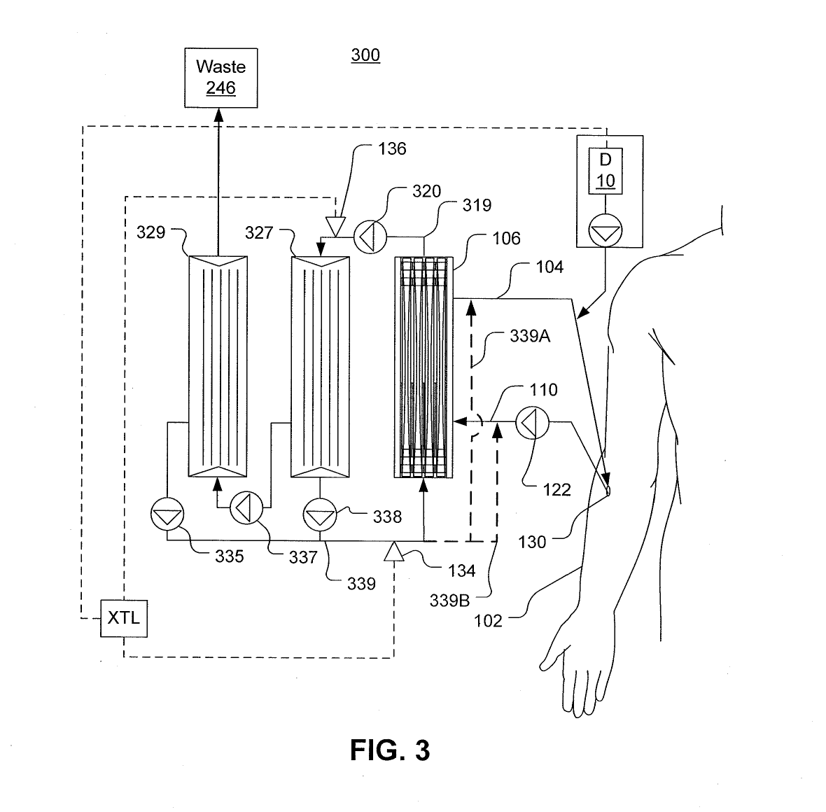

FIG. 3 shows an arrangement 300 for plasma extraction by microfluidic separation with middle size particle/molecule removal by cascade filtration. Blood is drawn from the patient 102 and passed through an arterial line 110 and into a microfluidic separation module 106 as described in the '117 application mentioned above. Blood is returned to the patient 102 via the venous line 104. The microfluidic separation module 106 removes a cytoplasmic-body-free fraction of the blood, exchanges components by diffusion, or a combination of the two. The cytoplasmic-body-free fraction of the blood passes through a supply line 319, impelled by a pump 320, into a filter cascade with large pore filter 327 and small pore filter 329. A target component is removed by extracting the filtrand after small components are filtered by filter 329 from the filtrate of filter 327. The filtrand of filter 327 and filtrate of filter 329 are recovered as the cleansed cytoplasmic-body-free fraction and returned to the microfluidic separation module 106 via a return line 339 driven by pumps 335 and 338. Substituate (not shown) may be supplied at appropriate points in the system to make up for lost fluid volume or priming as required. As described above, alternative embodiments of return lines 339A and 339B may be provided instead of return line 339.

As in preceding embodiments, detectors 134 and 136 detect an amount of a target substance in the supply and discharge lines 319 and 339, allowing a controller XTL to determine an amount of a target substance being removed. This may be done in near real time by a lab on a chip assay device, for example, or by some other type of sensor. The controller may use the rate of removal of the target to control the infusion pump 10 which infuses a drug or medicament into the venous line 104 and thereby into the patient 102. Examples of substances that may be infused and then recovered also include imaging contrast agents, diagnostic agents, and treatment drugs such as chemotherapeutic agents for cancer treatment.

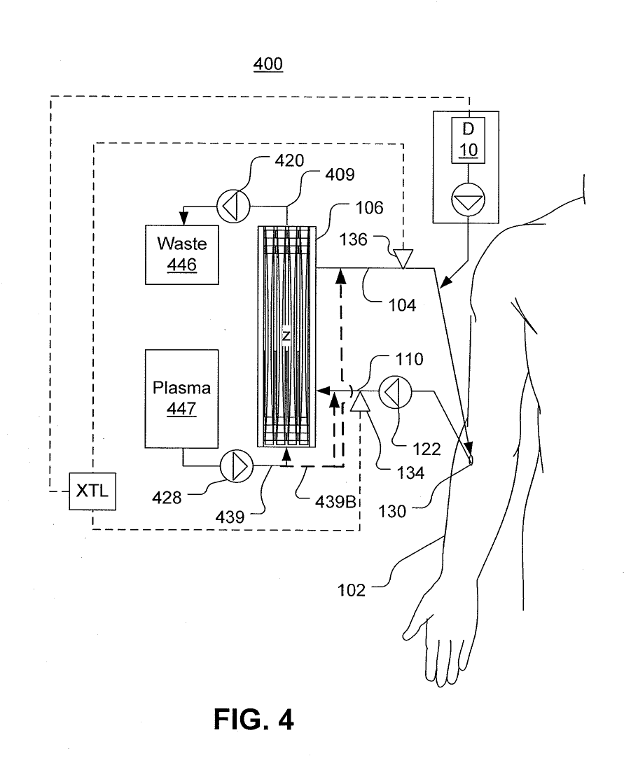

FIGS. 4 and 5 show arrangements 400 and 500 for toxin extraction by microfluidic separation with plasma replacement. In both systems 400 and 500, blood is drawn from the patient 102 and passed through an arterial line 110 and into a microfluidic separation module 106 as described in the '117 application. Blood is returned to the patient 102 via the venous line 104. The microfluidic separation module 106 removes a cytoplasmic-body-free fraction of the blood, exchanges components by diffusion, or a combination of the two. The cytoplasmic-body-free fraction of the blood passes through a supply line 409, impelled by a pump 420, where it is monitored and discarded 446. Replacement plasma 447 from an exogenous source is provided by a pump 428 to the microfluidic extractor 106 through a return line 439, which is monitored by the detector 134. Again, different return locations may be provided in alternative embodiments as indicated by return lines 439A and 439B. The alternative return line embodiments are not shown in further figures but it is understood that these variants are applicable to the all embodiments described.

As in preceding embodiments, detectors 134 and 136 can detect an amount of a target substance in the supply and discharge lines 409 and 439, or in the arterial line 110 and the venous line 104, allowing a controller XTL to determine an amount of a target substance being removed. This may be done in near real time by a lab on a chip assay device, for example, or by some other type of sensor. The controller may use the rate of removal of the target to control the infusion pump 10 which infuses a drug or medicament into the venous line 104 and thereby into the patient 102. Examples of substances that may be infused and then recovered also include imaging contrast agents, diagnostic agents, and treatment drugs such as chemotherapeutic agents for cancer treatment.

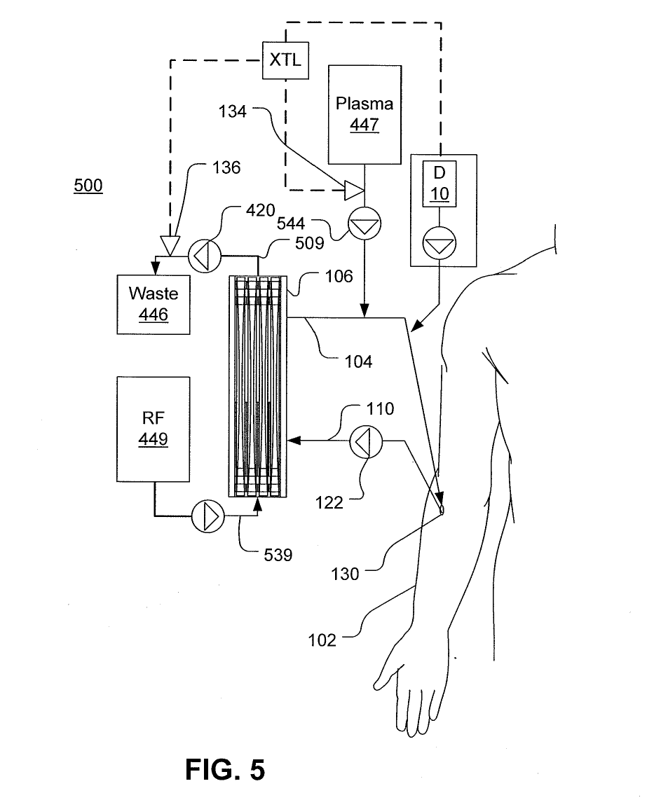

System 500 is substantially similar to system 400 except for the differences noted hereinbelow. Referring to FIG. 5, fresh plasma 447 is returned directly to the patient by an infusion pump 544. In this embodiment, a replacement fluid or medicament 449 may be provided to the microfluidic separation module 106, but a constant supply may not be required since, as discussed in the aforementioned patent-related documents corresponding to Appendices A-C of Provisional Application No. 61/325,765, the microfluidic separation module is able to remove plasma without an incoming fluid. In addition, a closed loop return from line 509 to 539 may be provided and this may be selectable as shown in FIG. 6. As in preceding embodiments, detectors 134 and 136 detect an amount of a target substance in the supply and discharge lines and the controller XTL implements a treatment prescription.

FIG. 6 illustrates a dialysis based plasma purification system 600 in which flow selector valves, controls, and other elements that are usable in any of the embodiments are employed to permit the closed loop cycling of cytoplasmic body-free blood fractions in a microfluidic separation module during infusion and treatment intervals in which blood is not being cleansed. The system 600 is substantially similar to that of system 100 in FIG. 1, except that it is provided with a short circuit bypass line 610 to bypass the filter 108 and selector valves 602 to 608 to divert the flow of plasma such that the system can run in a bypass mode circulating blood through the microfluidic separation module without processing the plasma, thereby ensuring patency of the access 130 and blood lines 104 and 110. This system may be useful where the treatment plan involves monitoring the patient response and it cannot be predicted when, or how quickly the plasma purification process may need to be implemented.

Examples of drugs that may be used in treatments (either alone or in conjunction with other drugs) as described are (i) drugs such as cisplatin, cyclophosphamide, docetaxel, doxorubicin, etoposide, idarubicin, lomustine, melphalan, paclitaxel and pemetrexed, which are highly protein-bound and have long half-life; and (ii) drugs such as busulfan, capecitibine, carmustine, temozolomide, thiotepa, vincristine and valrubicin, which are also protein-bound and have short but toxic half-life. Systems such as described above may be adapted to quantify an amount of each of multiple drugs added to and removed from the patient in a rapid process.

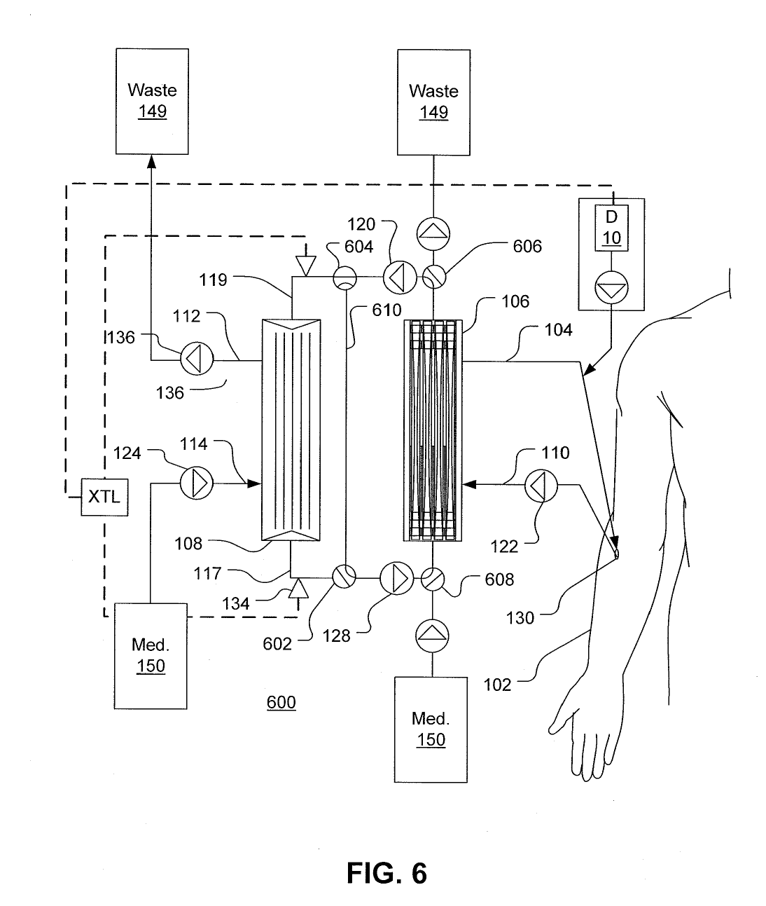

Referring to FIG. 7, in such systems for multiple drug administration and removal, rather than a single sensor for each of the in-going and out-going fluids (e.g., plasma or blood) separate drug-specific sensors 704, 706 may be provided to indicate a respective drug and generate a signal that may indicate the quantity of drug in a sampled fluid. The quantity may be converted to a rate such as net outtake of the drug from the system computationally by a programmable control unit 702 which may generate treatment information for output on a display 716 and/or control commands for execution by drug administering final controllers 712, 714 such as infusion pumps.

FIG. 8 shows an arrangement for plasma extraction by microfluidic separation with middle size particle/molecule removal by cascade filtration implementation of drug infusion without implementation of drug infusion. FIG. 8 is based on FIG. 3, but notably lacks infusion pump 10. Thus, the embodiment shown by FIG. 8 may not be used to infuse a drug or medicine into the patient 102. Rather, the embodiment shown by FIG. 8 may be used to separate an unknown and unwanted target component from the patient's blood stream. Unwanted target components may be characterized as classes and thus the embodiment of FIG. 8 can be used to selectively remove or reduce multiple whole classes of unwanted components, such as bacteria, viruses, activated patient cells, biomolecules, and toxins. Alternatively, embodiments may be used to remove only a single class of targets or a single target within a class. Optionally, use of molecular labels or specific binding chemistries is not implemented in the embodiment of FIG. 8 in order to remove unknown and unwanted target components.

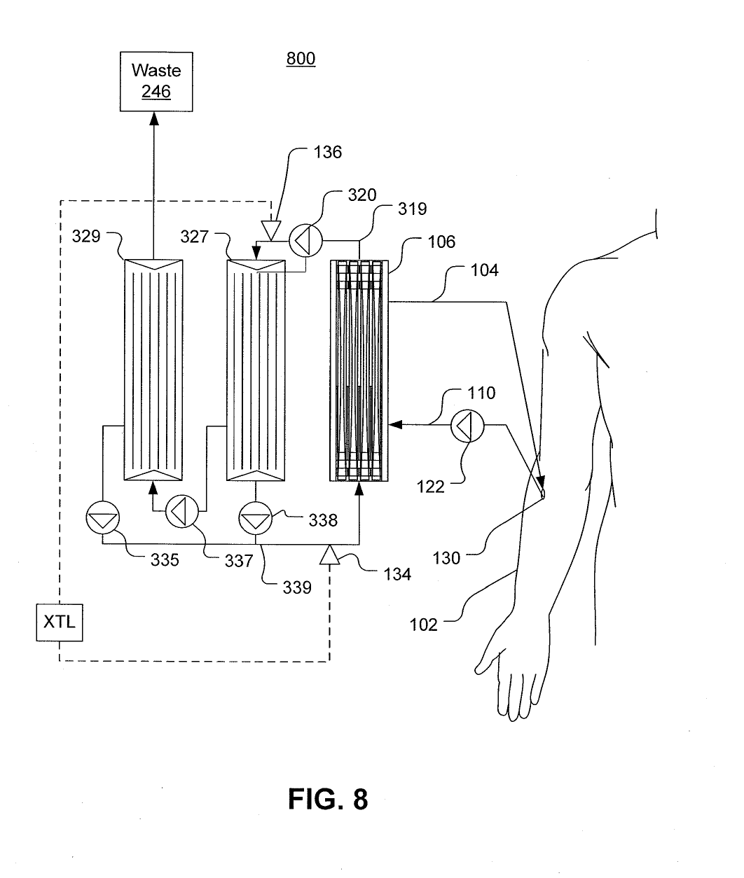

FIGS. 9A and 9B schematically illustrate the application of alternative cleansed plasma return line configurations with respect to the operation of the microfluidic separation module. Referring to FIG. 9A, in embodiments described above where cleansed plasma is returned to a patient arterial blood line (e.g., embodiment 129B shown in FIG. 1), cleansed plasma 812 may be mixed with blood 804 in the arterial line before entering the microfluidic separation module 802. The incoming blood 804 is thus diluted with cleansed plasma 814 before it enters the microfluidic channel 800. In this case, the structure of the microfluidic separation module 802 does not require a separate inlet thereby simplifying construction. Referring to FIG. 9B, the return line is returned to the patient venous line (e.g., embodiment 129A in FIG. 1), cleansed plasma 814 may be mixed with blood 806 after cleansing and returned thereafter to the patient. The structure of a microfluidic separation module is similarly simplified over structures disclosed in some incorporated references. Note the portions shown in 9A and 9B may be formed in a stack to create a high surface area module or a single channel may be used in a detector module.

FIG. 10 illustrates a method for treating a patient in a field environment such as a battlefield environment or a military field hospital. Initially, a patient is identified as being at risk of having or developing a condition involving an unwanted substance in the blood. For example, the patient may be a wounded soldier at risk of sepsis. The patient is connected S100 to a microfluidic separation module (MSM) and connected fluid circuit. The circuit may be pre-primed with blood normal sterile fluid or primed in a follow on step S102. Blood is pumped through the MSM while the blood is monitored for the presence of sepsis. For example, minute quantities of plasma may be separated from the blood continuously or intermittently and analyzed for the presence of indicators of sepsis using a suitable detector as described above. Alternatively, sepsis may be detected in a separate process in which blood plasma is not removed from the MSM until treatment is initiated at a later step. If sepsis is detected at S106, the plasma rate is increased or initiated depending on the embodiment as indicated at step S108. This step S108 subsumes any or a combination of the treatment embodiments described above including:
Filtering from the plasma small particles and albumin and replacing albumin to selectively remove bacteria particles and replace required component as needed from an exogenous source.
Filtering intermediate particles such as bacteria from the separated plasma using a filter cascade and returning small components and larger components in the plasma fraction back to the patient. This may include infusing precious components from an exogenous source.
Removing plasma and replacing with plasma from an exogenous source.
Infusing a drug such as an antibiotic.
Filtering selected target components (including an administered drug such as an antibiotic) using an adsorbent.

A combination of the above including intermittently interspersing removal and infusion of respective substances.

During step S108, blood and other patient conditions may be monitored for conditions indicating the use of a therapeutic agent such as an antibiotic. At step S110, the method determines whether a therapeutic agent is indicated and if so at step S112, the agent is infused. Step S114 monitors for the possible conditions and reverts or terminates the process accordingly.

In the method embodiment of FIG. 10, the device connected to the MSM patient may be small portable device which may allow for movement of the patient. For example, the MSM may remain connected along with a monitoring component and only connected to a secondary treatment stage at step S108. Such an embodiment would employ connectors, such as Luer connectors, to connect a closed loop of plasma to an expanded loop including the secondary treatment. The detection components as described herein may be located according to any of the configurations described with respect to the various embodiments.

Embodiments of the method of FIG. 10 may include or omit the infusion of anticoagulants.

Note that while according to the embodiments of FIG. 10, sepsis monitoring was performed with the MSM pre-attached, it is possible for sepsis monitoring to done separately and the MSM connected, primed, and immediately operated for treatment as at step S108.

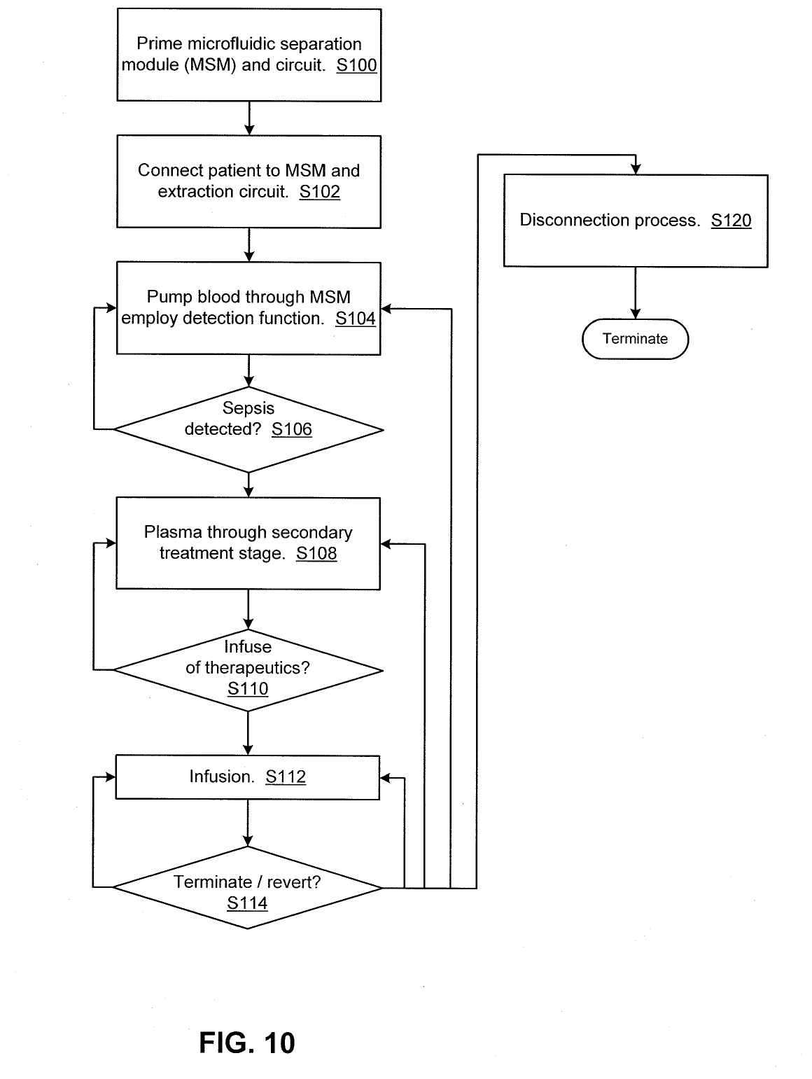

Referring to FIG. 11, embodiments may include detection and regulation of undesired blood components such as pathogen levels. These embodiments may also include detection and regulation of levels of therapeutic agents that change the levels of the unwanted substances and the regulation of the blood levels of the therapeutic agents themselves. Thus in all of the embodiments described herein, multiple types of detectors 1112 may be employed with a microfluidic separation module 1101. The detectors may be configured to detect various species. For example, one may indicate the presence of infection and another may indicate the blood levels of a treatment agent. The system may be configured with a controller 1107 to regulate the levels of both species. The latter capability may be provided by the use of combinations of treatment components 1103, 1105 (secondary treatment devices that remove target substances from extracted plasma) which are selectively switched in and out of the plasma loop 1114 by flow diverters d, for example, responsively to the detected levels of species in the blood or plasma.

PCT publication WO2011/025986 for “Multi-Layered Blood Component Exchange Devices, Systems, and Methods,” which is incorporated herein by reference in its entirety, describes details that are applicable for fabrication of the microfluidic separation module embodiments described throughout the present application and is hereby incorporated by reference in its entirety herein. According to the description the size of the microfluidic separation device can be scaled by stacking multiple channels as described in the reference. The result can achieve large interface area in a compact configuration which lends itself to a portable device.

In addition to drugs, treatments may also employ the administration of affinity agents for removal of viruses and/or virus proteins from the blood such as described in U.S. Pat. No. 7,226,429. The '429 patent describes removing pathogens bound to lectins which are filtered from the blood or plasma. In variations of the described embodiments, plasma may be separated from whole blood, treated, and returned to the patient. Other treatments are also possible such as described in U.S. Pat. No. 6,620,382 for removing large molecules in the treatment of cancer and U.S. Publication No. 2008/0138434 for treatment of infection by reducing the levels of pro- or anti-inflammatory stimulators or mediators such as cytokines using adsorption from plasma. The entire content of each of the aforementioned documents is hereby incorporated by reference into the present application. The embodiments may also be used in treatment systems where the circulation of an organ or other region of the body is isolated from the rest of a patient's circulatory system and high levels of drug infused into the organ's blood system and removed from the isolated flow.

Although most of the embodiments described employed adsorbent, deionization, and membrane filtration as mechanisms for removing substances from plasma, other mechanisms may be employed with the disclosed subject matter. For example, removal, modification, or destruction mechanisms may include exposing the target substance to a suitable electrical and/or magnetic field to discriminate, alter, or destroy the target substance. Optionally, the latter may include “labeling” target substances with magnetic or electrically polarized substances. Catalysis and/or enzyme reactions may be employed to modify or remove target substances.

In any of the embodiments described above, the microfluidic separation module may be omitted and whole blood passed directly through the various secondary separation components. These alternative embodiments are clearly enabled in the present disclosure though clearly not all features and benefits are provided by such alternatives.

Although particular configurations have been discussed herein, other configurations can also be employed. It is, thus, apparent that there is provided, in accordance with the present disclosure, filtration methods, devices, and systems. Many alternatives, modifications, and variations are enabled by the present disclosure. Features of the disclosed embodiments can be combined, rearranged, omitted, etc., within the scope of the invention to produce additional embodiments. Furthermore, certain features may sometimes be used to advantage without a corresponding use of other features. Accordingly, Applicants intend to embrace all such alternatives, modifications, equivalents, and variations that are within the spirit and scope of the present invention.

1. A method for extracorporeal treatment of blood to remove a target substance, comprising:
removing blood from a patient and passing the blood through a microfluidic channel, the microfluidic channel having microsieve wall filters therein;
extracting a cytoplasmic body-free blood fraction of the blood from the microfluidic channel by passing through the microsieve wall filters the a cytoplasmic body-free blood fraction including the target substance;
extracting at least some of the target substance from the removed cytoplasmic body-free fraction and returning the resulting cytoplasmic body-free fraction to the patient.

2. The method of claim 1, wherein the microsieve wall filters include microporous filters flush mounted in a wall of the microfluidic channel to form a continuous surface of the microfluidic channel.

3. The method of claim 1, wherein the microfluidic channel is a rectilinear microchannel having a depth across the flow of no more than 200 microns and a width at least ten times the depth.

4. The method of claim 1, wherein the extracting includes cascade filtration using multiple membranes having different pore sizes to select and filter out a target particle size range.

5. The method of claim 1, wherein the extracting includes adhering the target to an adsorbent.

6. The method of claim 1, further comprising monitoring an amount of the target substance in the body of a patient and controlling an administration of the target substance, or a precursor thereof, responsively to the monitoring.

7. The method any of claim 1, further comprising binding the target substance to another substance to form an aggregate particle, the extracting including extracting the aggregate particle.

8. The method of claim 7, wherein the aggregate particle has a higher binding affinity for an adsorbent and the extracting includes exposing the aggregate particle to the adsorbent.

9. The method of claim 1, further comprising monitoring an amount of the target substance in the body of a patient and controlling an administration of the target substance, or a precursor thereof, responsively to a predetermined time-concentration integral limit.

10. The method of claim 1, further comprising monitoring an amount of the target substance in the body of a patient and controlling an administration of the target substance, or a precursor thereof, responsively to predictive model of an elimination rate of the target substance from the patient by endogenous pathway(s).

11. The method of claim 1, wherein the patient has a faulty or sub-optimal endogenous elimination capacity, or where the target substance is toxic to the kidney, liver or other endogenous elimination routes.

12. The method of claim 1, wherein the target substance is a result of a drug overdose of clinical administration of a drug with low therapeutic index, high toxicity or long half-life either for a single dose (or treatment session) or cumulatively over the course of treatment.

13. The method of claim 1, wherein the monitoring includes sensing a level of at least one drug in the cytoplasmic body-free blood fraction.

14. A method for extracorporeal treatment of blood to remove a target substance, comprising:
removing blood from a patient and passing the blood through a microfluidic channel having wall filters with a diffusing the target substance into a cytoplasmic body-free blood fraction;
extracting and discarding the cytoplasmic body-free fraction from the microfluidic channel, the extracting including passing said fraction through a microsieve filter having a single pore size achieved by micromachining; and
returning fresh plasma to the patient.

15-33. (canceled)

34. A method for treating sepsis without use of anticoagulants, molecular labels, and/or specific binding chemistries, comprising:
drawing blood from a patient using a closed loop system;
detecting a particle in the drawn blood indicative of sepsis or oncoming sepsis using at least one non-fouling or substantially non-fouling sensor;
treating the drawn blood using a plurality of microfluidic separation channels to selectively remove the particle; and
returning to the patient cleaned blood having been subjected to said treating.

35. The method of claim 34, further comprising a plurality of said particles and said treating reduces or removes a portion of said plurality of said particles.

36. The method of claim 35, wherein the portion of reduced or removed particles is 90% of said plurality or greater, up to and including 100%.

37. The method of claim 36, wherein the 90% through 100% reduction or removal occurs within twenty-four hours.

38. The method of claim 34, wherein the flow rate of the closed loop system is 500 mL/hour or greater.

39. (canceled)

40. The method of claim 34, wherein the method is performed without platelet activation or clotting.

41-43. (canceled)
